(19) United States
(12) Patent Application Publication
(10) Pub. No.: US 2017/0112383 A1
(43) Pub. Date: Apr. 27, 2017

(54) THREE DIMENSIONAL VEIN IMAGING USING PHOTO-ACOUSTIC TOMOGRAPHY

(71) Applicant: NEC Laboratories America, Inc., Princeton, NJ (US)

(72) Inventors: Giovanni Milione, Franklin Square, NY (US); Philip Nan Ji, Cranbury, NJ (US); Ting Wang, West Windsor, NJ (US); Thien An Nguyen, Corning, NY (US)

(21) Appl. No.: 15/271,031

(22) Filed: Sep. 20, 2016

Related U.S. Application Data
(60) Provisional application No. 62/245,498, filed on Oct. 23, 2015.

(51) Int. Cl. A61B 5/00; A61B 5/117; A61B 5/02

(52) U.S. Cl. CPC A61B 5/0095; A61B 5/02007; A61B 5/117; A61B 5/7405; A61B 5/742; A61B 5/7475; G06K 9/00885

(57) ABSTRACT

Systems and methods for imaging a three-dimensional (3D) biometric object, including receiving the 3D biometric object into a scanning area. An image of the 3D biometric object is generated using photo-acoustic tomography by illuminating one or more veins in the 3D biometric object using a light source, and detecting acoustic waves resulting from absorption of light from the light source by the one or more veins. A 3D vein image is output based on the acoustic waves detected.

### RELATED APPLICATION INFORMATION

This application claims priority to provisional application No. 62/245,498 filed Oct. 23, 2015, the contents of which is incorporated herein by reference.

### BACKGROUND

Technical Field

The present invention relates generally to three-dimensional vein imaging, and more particularly, to three dimensionally imaging finger veins using photoacoustic tomography for three-dimensional biometrics.

Description of the Related Art

Biometrics is the exploitation of a human's unique anatomical features (e.g., voice, face, iris, finger-prints, finger-veins, etc.) to verify their identity. Unlike, for example, passwords or driver's licenses, it is more difficult to falsify anatomical features. Due to the exponential growth of data-driven technologies, biometrics is becoming a ubiquitous technology for applications that require increasingly high-levels of security via identity verification, including, for example, immigration control, law enforcement, personal banking, and ticket monitoring for recreational businesses (e.g. Disneyland®).

For example, obtaining two-dimensional biometrics by two-dimensionally imaging finger-veins is an attractive biometric as it meets salient biometric requirements including, for example, universality, distinctiveness, permanence, collectability, performance, acceptability, and low circumvention. Finger-veins can be two-dimensionally imaged by illuminating a finger with light at such a wavelength (e.g. near infrared) that it is absorbed by the blood in the finger-veins. The scattered light is then detected by a camera to produce a two-dimensional finger-vein image where the finger-veins appear as a two-dimensional network of dark lines. Used as two-dimensional biometrics, two-dimension finger-vein images can be digitally processed for identity verification. Using two-dimensional finger-vein images as two-dimensional biometrics provides low false reject rates (e.g., the frequency with which genuine identity is not correctly recognized), and low false accept rates (e.g., the frequency with which an imposter is accepted as genuine identity).

However, as compared to two-dimensional biometrics, three-dimensional biometrics are more robust to illumination, provide a richer information source for feature extraction, and are more difficult to falsify. In turn, three-dimensional biometrics provide lower false reject rates and lower false accept rates than two-dimensional biometrics. Finger-veins have a three-dimensional structure. As finger veins exist just below the finger surface their three-dimensional structure is due to the curvature of the finger surface and their depth-dependent structure with respect to the finger surface. However, it is challenging to three-dimensionally image finger-veins. This is at least in part due to the inherent increase of deleterious light scattering as light propagates deeper into biological tissue (e.g., a finger), which in turn degrades image quality.

Conventionally, finger-veins have been three-dimensionally imaged by using two cameras positioned at two different viewpoints. This results in disparate finger-vein images comprising a three-dimensional image. However, the resulting three-dimensional image is of the structure of finger veins due to the curvature of the finger surface; it obtains an image of finger-veins just below the finger surface, and does not overcome at least the problem of deleterious light scattering. Due to deleterious light scattering, it is not possible to obtain accurate three-dimensional finger-vein images associated with their depth-dependent structures using conventional systems and methods.

### SUMMARY

A computer implemented method for imaging a three-dimensional (3D) biometric object, including receiving the 3D biometric object into a scanning area. An image of the 3D biometric object is generated using photo-acoustic tomography by illuminating one or more veins in the 3D biometric object using a light source, and detecting acoustic waves resulting from absorption of light from the light source by the one or more veins. A 3D vein image is output based on the acoustic waves detected.

A system for imaging a three-dimensional (3D) biometric object, including a scanning area for receiving the 3D biometric object for imaging. An image generator is configured to generate an image of the 3D biometric object using photo-acoustic tomography by illuminating one or more veins in the 3D biometric object using a light source. Acoustic waves resulting from absorption of light from the light source by the one or more veins are detected using an acoustic wave detector, and a 3D vein image is output based on the acoustic waves detected.

A computer-readable storage medium including a computer-readable program for imaging a three-dimensional (3D) biometric object, including receiving the 3D biometric object into a scanning area. An image of the 3D biometric object is generated using photo-acoustic tomography by illuminating one or more veins in the 3D biometric object using a light source, and detecting acoustic waves resulting from absorption of light from the light source by the one or more veins. A 3D vein image is output based on the acoustic waves detected.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a block/flow diagram illustrating an exemplary processing system to which the present principles may be applied, in accordance with the present principles;

FIG. 2A is an diagram illustrating an exemplary human finger and finger veins, in accordance with an embodiment of the present principles;

FIG. 2B is a diagram illustrating a system/method for three-dimensional imaging of finger veins by illuminating finger veins, in accordance with an embodiment of the present principles;

FIG. 2C is a diagram illustrating a system/method for three-dimensional imaging of finger veins using photo-acoustic tomography for three-dimensional biometrics, in accordance with an embodiment of the present principles;

FIG. 3A is a diagram illustrating a system/method for three-dimensional imaging of finger veins using photo-acoustic tomography including a light source with spatial resolution, in accordance with an embodiment of the present principles;

FIG. 3B is a diagram illustrating a system/method for three-dimensional imaging of finger veins using photo-acoustic tomography including an acoustic wave detector with spatial resolution, in accordance with an embodiment of the present principles;

FIG. 3C is a diagram illustrating a system/method for three-dimensional imaging of finger veins using photo-acoustic tomography including a raster scanned light source and a fixed position acoustic wave detector, in accordance with an embodiment of the present principles;

FIG. 3D is a diagram illustrating a system/method for three-dimensional imaging of finger veins using photo-acoustic tomography including a fixed position light source and a raster scanned acoustic wave detector, in accordance with an embodiment of the present principles;

FIG. 4A is a diagram illustrating a system/method for three-dimensional imaging of finger veins using photo-acoustic tomography including a light source and an acoustic wave detector positioned on a same side of a finger, in accordance with an embodiment of the present principles;

FIG. 4B is a diagram illustrating a system/method for three-dimensional imaging of finger veins using photo-acoustic tomography including a light source and an acoustic wave detector positioned on opposite sides of a finger, in accordance with an embodiment of the present principles;

FIG. 4C is a diagram illustrating a system/method for three-dimensional imaging of finger veins using photo-acoustic tomography including a light source and an acoustic wave detector positioned at a right angle to each other, in accordance with an embodiment of the present principles;

FIG. 5 is a block/flow diagram illustrating a method for three-dimensional imaging of veins using photo-acoustic tomography for three-dimensional biometrics, in accordance with an embodiment of the present principles; and

FIG. 6 is a block/flow diagram illustrating a system for three-dimensional imaging of veins using photo-acoustic tomography for three-dimensional biometrics, in accordance with an embodiment of the present principles.

### DETAILED DESCRIPTION OF PREFERRED EMBODIMENTS

In accordance with various embodiments of the present principles, systems and methods are provided for three-dimensional imaging of finger veins using photo-acoustic tomography for three-dimensional (3D) biometrics.

In a particularly useful embodiment, the present principles may be employed to, for example, verify a person's identity (e.g., biometric lock, banking, etc.) by performing 3D imaging of finger veins using photo acoustic tomography to obtain 3D biometrics in accordance with the present principles.

In one embodiment, the present principles may be employed to obtain three-dimensional (3D) biometrics by three-dimensional imaging of human veins (e.g., finger-veins) using photo-acoustic tomography. In photo-acoustic tomography, biological tissue is illuminated with pulsed light at such a wavelength that it is absorbed. The absorption of the light results in acoustic waves. As acoustic waves experience significantly less deleterious scattering in biological tissue than light, the present principles may be employed to three-dimensionally image veins (e.g., finger-veins) using photo-acoustic tomography to obtain three-dimensional biometrics in accordance with various embodiments.

In one embodiment, instead of obtaining an image of the biological tissue by detecting scattered light with a camera, an image of the biological tissue can be obtained by detecting acoustic waves. The light source and/or the acoustic wave detector may be given spatial resolution in all three Cartesian directions (e.g. x, y, z). Three-dimensional finger vein images can be tomographically constructed. Using photo-acoustic tomography to three dimensionally image finger-veins for use as three-dimensional biometrics solves the problem of deleterious light scattering described above in accordance with the present principles.

The three-dimensional biometrics obtained by three-dimensional imaging of finger-veins using photo-acoustic tomography in accordance with the present principles are of higher-quality (e.g., more accurate, less false positives/negatives, etc.) than other biometrics. This is at least in part because three-dimensionally imaging finger-veins by photo-acoustic imaging meets salient biometric requirements including, universality, distinctiveness, permanence, collectability, performance, acceptability, and low circumvention, and additionally has the benefits of three-dimensional biometrics (e.g., being more robust to illumination, providing a richer information source for feature extraction, and being more difficult to falsify, etc.), which achieves lower false reject rates and lower false accept rates than conventional systems and methods.

As the exponential growth of data-driven technologies will require increasingly high-levels of security via identity verification, the higher-quality biometrics provided by three-dimensionally imaging finger-veins using photo-acoustic tomography according to the present principles may be employed for a plurality of purposes (e.g., identity verification for immigration control, law enforcement, personal banking, ticket monitoring, etc. according to various embodiments.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid state memory, magnetic tape, a removable computer diskette, a random access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

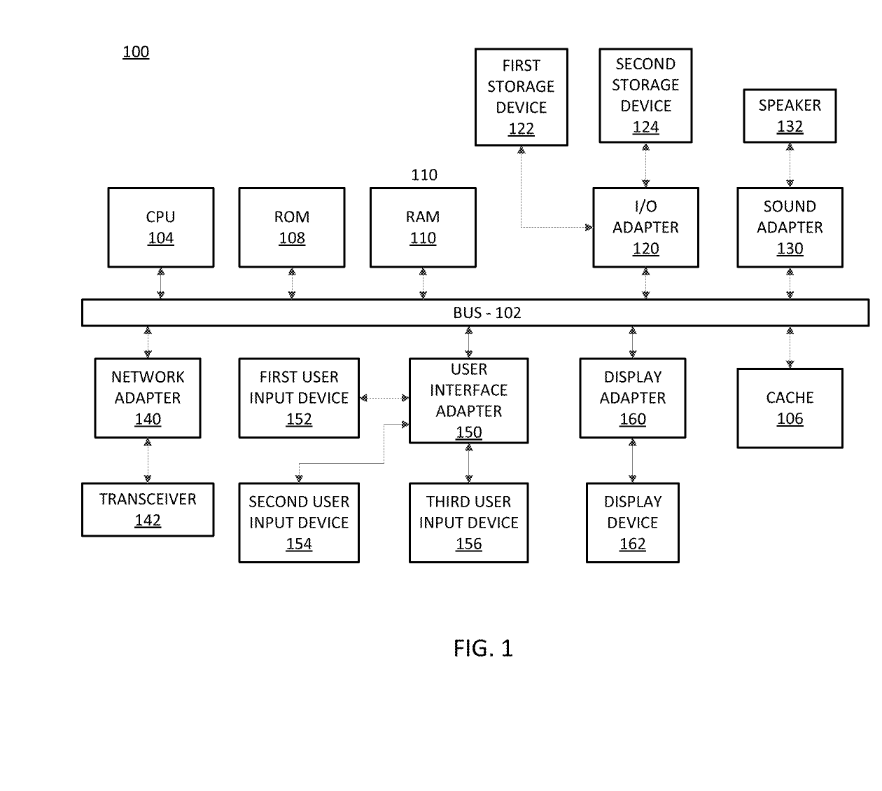

Referring now to the drawings in which like numerals represent the same or similar elements and initially to FIG. 1, an exemplary processing system 100, to which the present principles may be applied, is illustratively depicted in accordance with an embodiment of the present principles. The processing system 100 includes at least one processor (CPU) 104 operatively coupled to other components via a system bus 102. A cache 106, a Read Only Memory (ROM) 108, a Random Access Memory (RAM) 110, an input/output (I/O) adapter 120, a sound adapter 130, a network adapter 140, a user interface adapter 150, and a display adapter 160, are operatively coupled to the system bus 102.

A first storage device 122 and a second storage device 124 are operatively coupled to system bus 102 by the I/O adapter 120. The storage devices 122 and 124 can be any of a disk storage device (e.g., a magnetic or optical disk storage device), a solid state magnetic device, and so forth. The storage devices 122 and 124 can be the same type of storage device or different types of storage devices.

A speaker 132 is operatively coupled to system bus 102 by the sound adapter 130. A transceiver 142 is operatively coupled to system bus 102 by network adapter 140. A display device 162 is operatively coupled to system bus 102 by display adapter 160.

A first user input device 152, a second user input device 154, and a third user input device 156 are operatively coupled to system bus 102 by user interface adapter 150. The user input devices 152, 154, and 156 can be any of a keyboard, a mouse, a keypad, an image capture device, a motion sensing device, a microphone, a device incorporating the functionality of at least two of the preceding devices, and so forth. Of course, other types of input devices can also be used, while maintaining the spirit of the present principles. The user input devices 152, 154, and 156 can be the same type of user input device or different types of user input devices. The user input devices 152, 154, and 156 are used to input and output information to and from system 100.

Of course, the processing system 100 may also include other elements (not shown), as readily contemplated by one of skill in the art, as well as omit certain elements. For example, various other input devices and/or output devices can be included in processing system 100, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized as readily appreciated by one of ordinary skill in the art. These and other variations of the processing system 100 are readily contemplated by one of ordinary skill in the art given the teachings of the present principles provided herein.

Moreover, it is to be appreciated that systems 100, 200, 300, 400, and 600, described with respect to FIGS. 1, 2, 3, 4, and 6, respectively, are systems for implementing respective embodiments of the present principles. Part or all of processing system 100 may be implemented in one or more of the elements of systems 200, 300, 400, and 600, according to various embodiments of the present principles.

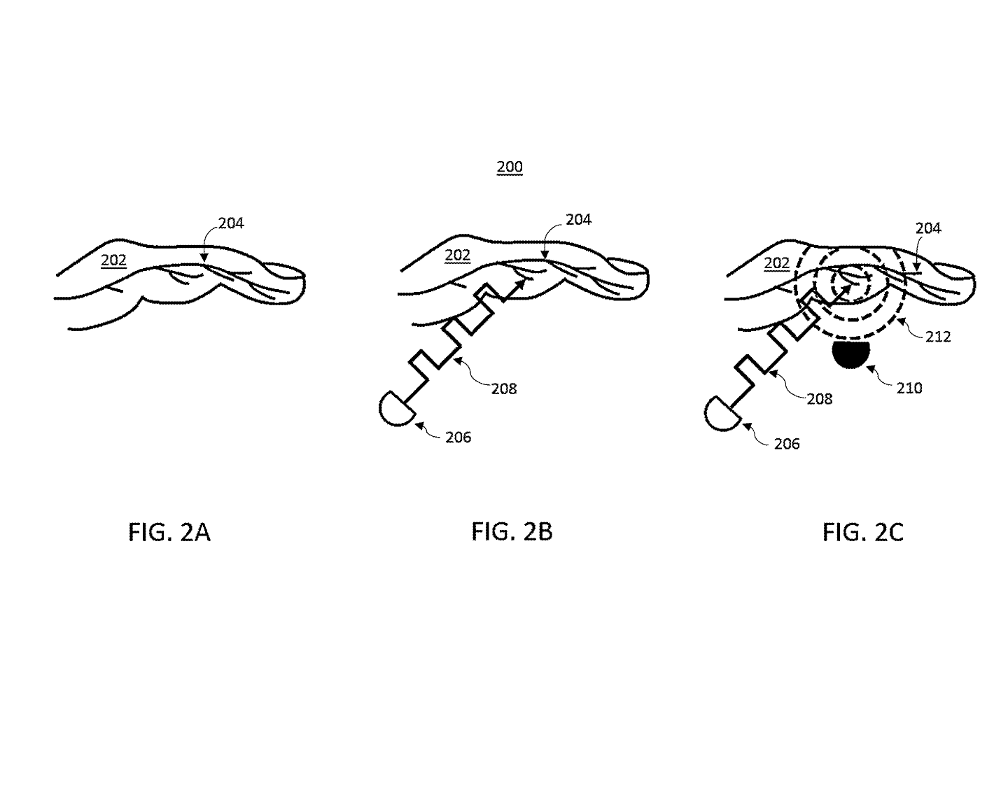

Further, it is to be appreciated that processing system 100 may perform at least part of the method described herein including, for example, at least part of methods 200, 300, 400, and 500 of FIGS. 2, 3, 4, and 5. Similarly, part or all of systems 200, 300, 400, and 600 may be used to perform at least part of methods 200, 300.400, and 500 of FIGS. 2, 3, 4, and 5 according to various embodiments of the present principles.

Referring now to FIG. 2A, an exemplary diagram of a human finger 202 and finger veins 204 to be imaged is illustratively depicted in accordance with the present principles. Finger veins 204 in a human finger 202 have a three-dimensional (3D) structure. As finger veins 204 exist just below the finger 202 surface, the 3D structure of finger veins 204 is due to the curvature of the finger 204 surface and their depth-dependent structure with respect to the finger 204 surface.

Although the present principles are described with respect to human finger vein imaging, it is to be appreciated that the present principles may be applied to other veins and parts of a body in accordance with various embodiments of the present principles.

Referring now to FIG. 2B, a diagram of a system/method 200 for three-dimensional imaging of finger veins 204 by illuminating finger veins 204 in a human finger 202 is illustratively depicted in accordance with the present principles. In one embodiment, finger veins 204 may be illuminated with light 208 (e.g., light pulse) from a light source 206 (e.g., laser, non-laser light, etc.) at such a wavelength (e.g., near infrared) that it is absorbed by the blood in the finger veins 204. The light may be temporally modulated/pulsed (e.g., turned on and off over a period of time) in accordance with the present principles. In some embodiments, the light source 206 may include, for example, a pulsed laser, a temporally modulated continuous wave laser, a temporally modulated non-laser, etc. in accordance with the present principles.

Referring now to FIG. 2C, a diagram of a system/method 200 for three-dimensional imaging of veins 204 in a finger 202 using photo-acoustic tomography including an acoustic wave detector 210 with spatial resolution is illustratively depicted in accordance with the present principles. In one embodiment, absorption of pulsed light 208 from a light source 206 by the blood in the finger-veins results in an acoustic wave 212 whose amplitude may be measured to be directly proportional to the amount of light that is absorbed. As compared to light, acoustic waves experience significantly less deleterious scattering in biological tissue.

By detecting the acoustic waves 212 in accordance with the present principles, a three-dimensional image of the finger-veins can be obtained. In accordance with various embodiments, the acoustic wave detector 212 may be configured in several positions in relation to a finger to be imaged. For example, the acoustic wave detector 212 may be in contact with the finger 202 (e.g., acoustic transducer), or in non-contact with the finger 202 (e.g., optical interferometry) in accordance with the present principles.

In some embodiments, the light source 206 and/or the acoustic wave detector 212 may be given spatial resolution in all three Cartesian directions (e.g. x, y, z). Three-dimensional finger vein images may be tomographically constructed using the light source 206 and the acoustic wave detector 212, and the resulting three-dimensional finger-vein image may be employed as a three-dimensional biometric (e.g., for identity verification) in accordance with the present principles.

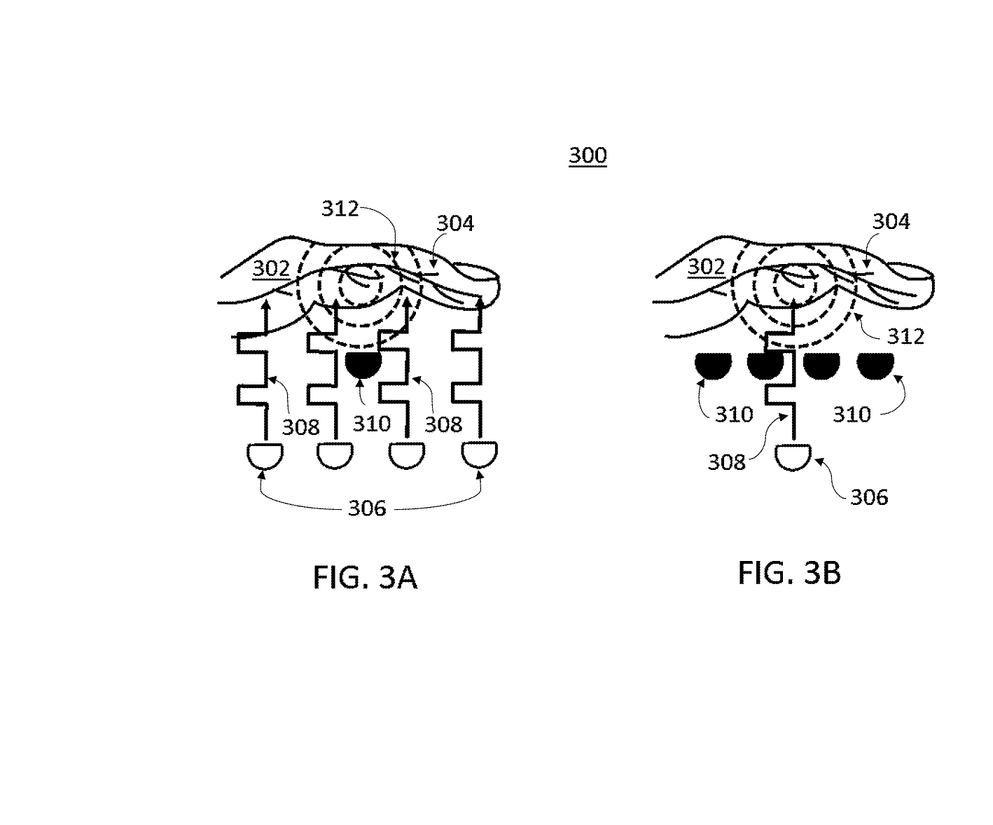

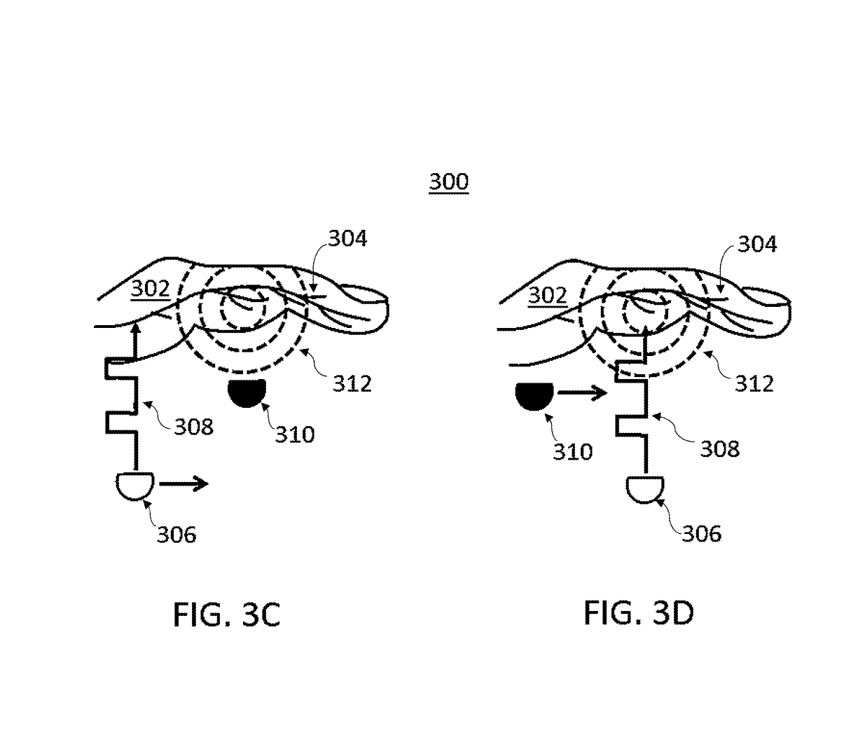

Referring now to FIG. 3A, a diagram of a system/method 300 for three-dimensional imaging of veins 304 in a finger 302 using photo-acoustic tomography including a light source 306 with spatial resolution and an acoustic wave detector 310 with a fixed position (e.g., no spatial resolution) is illustratively depicted in accordance with the present principles.

In various embodiments, spatial resolution may be achieved for three-dimensionally image finger veins 304 in accordance with the present principles. The light source 306 and/or acoustic wave detector 310 may be given spatial resolution in all three Cartesian directions (e.g., x, y, z). Three dimensional finger vein images may then be tomographically constructed according to various embodiments of the present principles, which are described in further detail with reference to FIGS. 3A-3D. Effectively, tomographic reconstruction comprises triangulation of the acoustic wave sources. Triangulation is accomplished by using the speed of sound to determine the time-of-flight of the generated acoustic waves outside the finger when they are recorded at multiple locations. Then, using an inverse reconstruction algorithm, the time-of-flights are converted into depth information thereby providing a 3D finger vein image

In one embodiment, absorption of pulsed light 308 from a light source 306 by the blood in the finger-veins 304 results in an acoustic wave 312 whose amplitude may be measured to be directly proportional to the amount of light 308 that is absorbed.

In some embodiments including a light source 306 with spatial resolution, the light 308 may be provided by, for example, by a digital micro-mirror device or a spatial light modulator. The acoustic wave detector 310 with a fixed position (e.g., no spatial resolution), may be, for example, a single acoustic transducer employed in accordance with various embodiments of the present principles. A three-dimensional biometric image may be generated using photoacoustic tomography based on the measured acoustic wave 312 in accordance with the present principles.

Referring now to FIG. 3B, a diagram of a system/method 300 for three-dimensional imaging of veins 304 in a finger 302 using photo-acoustic tomography including a light source 306 with a fixed position (e.g., no spatial resolution) and an acoustic wave detector 310 with spatial resolution (e.g., not fixed position) is illustratively depicted in accordance with the present principles. In one embodiment, absorption of pulsed light 308 from a light source 306 by the blood in the finger-veins 304 results in an acoustic wave 312 whose amplitude may be measured to be directly proportional to the amount of light 308 that is absorbed.

In some embodiments including a light source 306 with fixed position (e.g., no spatial resolution), and an acoustic wave detector 310 with spatial resolution (e.g., not fixed position), the acoustic wave detector 310 may include, for example, an array of acoustic transducers 310 employed in accordance with various embodiments of the present principles. A three-dimensional biometric image may be generated using photoacoustic tomography based on the measured acoustic wave 312 in accordance with the present principles.

Referring now to FIG. 3C, a diagram of a system/method 300 for three-dimensional imaging of veins 304 in a finger 302 using photo-acoustic tomography including a raster scanned light source 306 and an acoustic wave detector 310 with a fixed position (e.g., no spatial resolution) is illustratively depicted in accordance with the present principles. In one embodiment, absorption of pulsed light 308 from a light source 306 by the blood in the finger-veins 304 results in an acoustic wave 312 whose amplitude may be measured to be directly proportional to the amount of light 308 that is absorbed.

In one embodiment, the light source 306 does not have spatial resolution, but the light source 306 may be raster scanned in accordance with the present principles. The acoustic wave detector 310 may have a fixed position (e.g., no spatial resolution), and may be, for example, a single acoustic transducer 310 employed in accordance with the present principles. A three-dimensional biometric image may be generated using photoacoustic tomography based on the measured acoustic wave 312 in accordance with the present principles.

Referring now to FIG. 3D, a diagram of a system/method 300 for three-dimensional imaging of veins 304 in a finger 302 using photo-acoustic tomography including a fixed position light source 306 (e.g., no spatial resolution) and a raster scanned acoustic wave detector 310 is illustratively depicted in accordance with the present principles. In one embodiment, absorption of pulsed light 308 from a light source 306 by the blood in the finger-veins 304 results in an acoustic wave 312 whose amplitude may be measured to be directly proportional to the amount of light 308 that is absorbed.

In one embodiment, the light source 306 has a fixed position (e.g., no spatial resolution), and the acoustic wave detector 310 may not have spatial resolution, but the acoustic wave detector 310 may be raster scanned in accordance with the present principles. The light source 306 may be, for example, a laser 306, and the acoustic wave detector 310 may be, for example, a single acoustic transducer 310 employed in accordance with the present principles. A three-dimensional biometric image may be generated using photoacoustic tomography based on the measured acoustic wave 312 in accordance with the present principles.

Although the above configurations (e.g., spatial resolution, no spatial resolution, raster scanning) of the light source 306 and acoustic wave detector 310 are presented for illustrative purposes, it is to be appreciated that other configurations of the light source 306 and acoustic wave detector 310 (e.g., light source 306 and acoustic wave detector 310 both having spatial resolution, etc.) may be employed in accordance with various embodiments of the present principles.

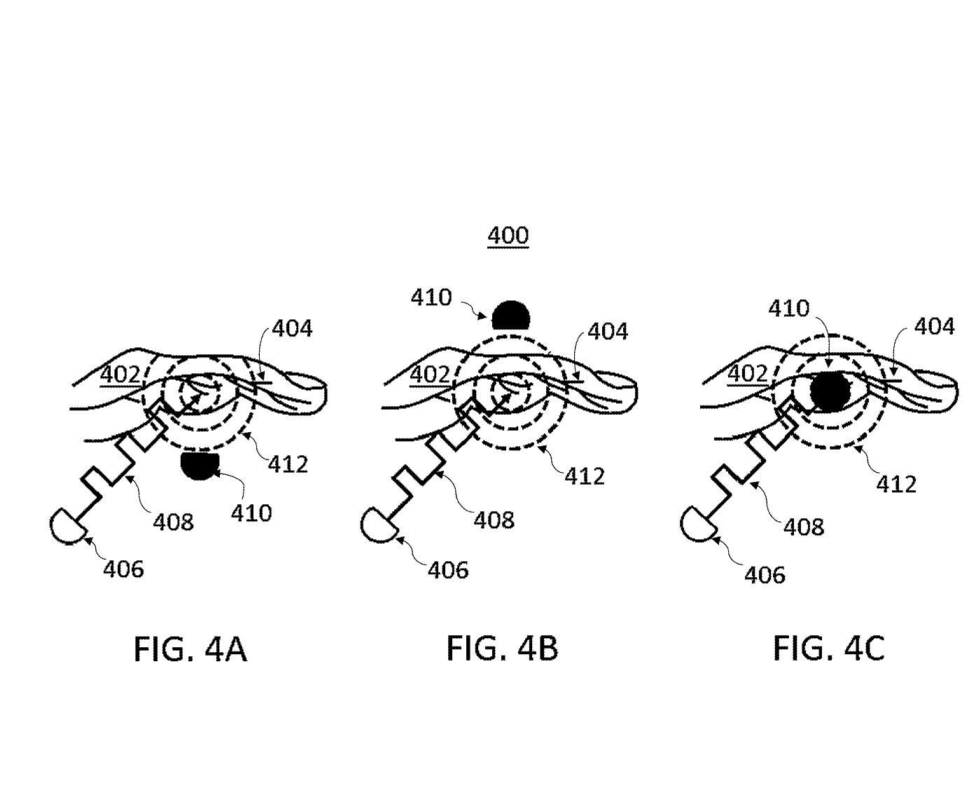

Referring now to FIG. 4A, a diagram of a system/method 400 for three-dimensional imaging of veins 404 in a finger 402 using photo-acoustic tomography is illustratively depicted in accordance with an embodiment of the present principles.

In one embodiment, a light source 406 and an acoustic wave detector 410 may be positioned on a same side of a finger 402 during photoacoustic imaging in accordance with the present principles. With this configuration, a user can place their finger on top of the system. Such a configuration, as opposed to other positions (e.g., when the light source and detector are on different sides of the finger, is advantageous because it can be used with, for example, a smart phone (e.g. iPhone), computer mouse, etc. Other configurations may not be equally beneficial, depending on a type of user equipment being employed (e.g., placing a light source and detector on different sides of a finger may be incompatible with compact devices, such as, smart phones). Absorption of pulsed light 408 from a light source 406 by the blood in the finger-veins 404 results in an acoustic wave 412 whose amplitude may be measured to be directly proportional to the amount of light 408 that is absorbed. A three-dimensional biometric image may be generated using photoacoustic tomography based on the measured acoustic wave 412 in accordance with the present principles.

Referring now to FIG. 4B, a diagram of a system/method 400 for three-dimensional imaging of veins 404 in a finger 402 using photo-acoustic tomography is illustratively depicted in accordance with an embodiment of the present principles.

In one embodiment, a light source 406 and an acoustic wave detector 410 may be positioned on opposite sides of a finger 402 during photoacoustic imaging in accordance with the present principles. Absorption of pulsed light 408 from a light source 406 by the blood in the finger-veins 404 results in an acoustic wave 412 whose amplitude may be measured to be directly proportional to the amount of light 408 that is absorbed. A three-dimensional biometric image may be generated using photoacoustic tomography based on the measured acoustic wave 412 in accordance with the present principles.

Referring now to FIG. 4C, a diagram of a system/method 400 for three-dimensional imaging of veins 404 in a finger 402 using photo-acoustic tomography is illustratively depicted in accordance with an embodiment of the present principles.

In one embodiment, a light source 406 and an acoustic wave detector 410 may be positioned at a right angle to each other during photoacoustic imaging in accordance with the present principles. Absorption of pulsed light 408 from a light source 406 by the blood in the finger-veins 404 results in an acoustic wave 412 whose amplitude may be measured to be directly proportional to the amount of light 408 that is absorbed. A three-dimensional biometric image may be generated using photoacoustic tomography based on the measured acoustic wave 412 in accordance with the present principles.

Although the above positioning of the light source 406 and acoustic wave detector 410 are presented for illustrative purposes, it is to be appreciated that other configurations including varying positions of the light source 406 and acoustic wave detector 410 may be employed in accordance with various embodiments of the present principles.

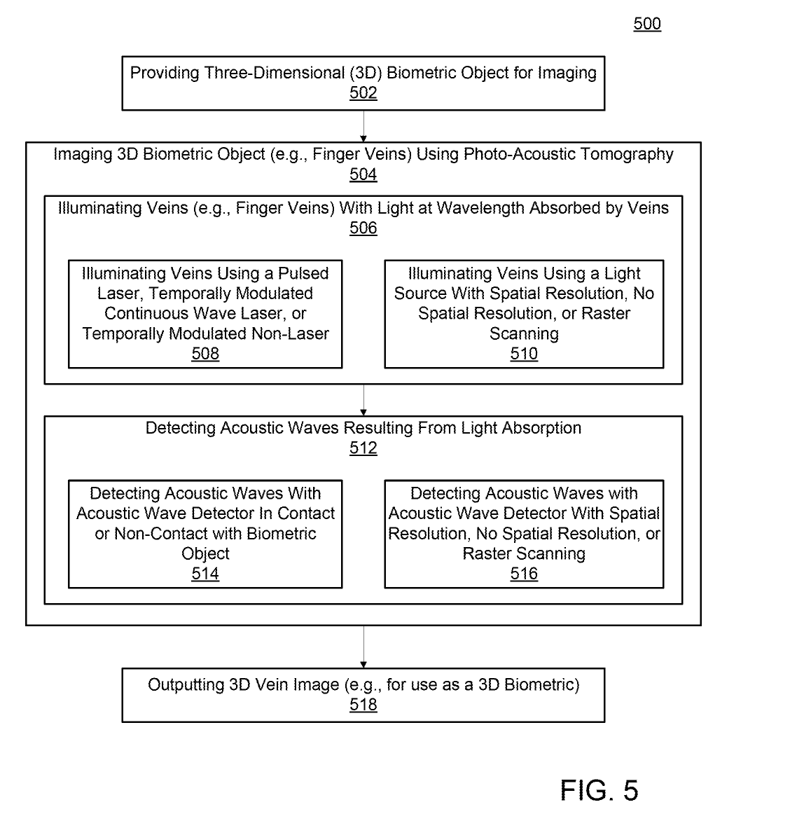

Referring now to FIG. 5, a block/flow diagram of a method 500 for three-dimensional imaging of finger veins using photo-acoustic tomography for three-dimensional biometrics is illustratively depicted in accordance with an embodiment of the present principles. In one embodiment, the method 500 may employ photo-acoustic tomography to three-dimensionally image finger-veins to overcome issues such as deleterious light scattering which occurs using conventional imaging systems and methods. The method 500 including performing photo-acoustic tomography according to the present principles results in lower false accept rates, lower false reject rates, and an overall higher-quality biometric than when using conventional methods.

Conventionally, finger-veins are three-dimensionally imaged by using two cameras positioned at two different viewpoints. This results in disparate finger-vein images comprising a three-dimensional image. However, the resulting three-dimensional image is of the structure of finger veins due to the curvature of the finger surface, as it obtains an image of finger-veins just below the finger surface. This method suffers from the problem of deleterious light scattering. Due to deleterious light scattering, it is exceedingly more difficult to obtain three-dimensional finger-vein images associated with their depth-dependent structure using conventional methods.

In one embodiment, a 3D biometric object (e.g., human finger) may be provided for imaging in block 502. In block 504, a 3D biometric object (e.g., finger veins) may be imaged using photo-acoustic tomography in accordance with the present principles. In block 508, finger-veins may be illuminated with light (e.g., pulsed light) at such a wavelength that it is absorbed by the blood of the finger-veins (e.g., near-infrared light having wavelengths from 600 nanometers to 1000 nanometers, infrared light having wavelengths from 1000 nanometers to 2000 nanometers, which generates an acoustic wave. The light source may be, for example, a pulsed laser, a temporally modulated continuous wave laser, or a temporally modulated non-laser in accordance with the present principles. In block 510, the illuminating light source may include a light source with spatial resolution, with a fixed position (e.g., no spatial resolution), or raster scanning in accordance with various embodiments.

In one embodiment, acoustic waves resulting from light absorption may be detected in block 512, and the acoustic waves may be detected with an acoustic wave detector in contact or in non-contact with the biometric object (e.g., finger) in block 514. In block 516, the acoustic waves may be detected using an acoustic wave detector with spatial resolution, with a fixed position (e.g., no spatial resolution), or raster scanning in accordance with various embodiments. In block 518, a 3D vein image of the biometric object (e.g., finger veins) may be generated by detecting the resulting acoustic waves, and output for use as a 3D biometric (e.g., in identity verification systems) in accordance with various embodiments.

In various embodiments, the illuminating light source in block 506 and the detecting acoustic wave detector in block 512 may be positioned, for example, on a same side of a finger, opposite sides, or at a right angle in accordance with the present principles. Although exemplary positioning of the light source and/or finger have been presented for illustrative purposes, it is to be appreciated that the light source may be positioned in any way such that an acoustic wave is produced in accordance with various embodiments of the present principles. Acoustic waves experience significantly less deleterious light scattering as compared to light waves, and as such, the method 500 solves the problem of deleterious light scattering. The three-dimensional finger-vein image can be used as a three-dimensional biometric in accordance with the present principles.

An advantage achieved by the method 500 of obtaining three-dimensional biometrics by three-dimensional imaging of finger-veins using photo-acoustic tomography according to the present principles is that the resulting image is of higher-quality than conventional biometrics. This is at least in part because three-dimensionally imaging finger-veins by photo-acoustic imaging meets salient biometric requirements including, for example, universality, distinctiveness, permanence, collectability, performance, acceptability, and low circumvention, and additionally has the benefits of three-dimensional biometrics (e.g., being more robust to illumination, providing a richer information source for feature extraction, and being more difficult to falsify), in turn achieving lower false reject rates and lower false accept rates. As the exponential growth of data-driven technologies will require increasingly high-levels of security via identity verification, the higher-quality biometrics provided by three-dimensionally imaging finger-veins using photo-acoustic tomography according to the present principles will provide a competitive and commercial value for a plurality of industries in accordance with various embodiments.

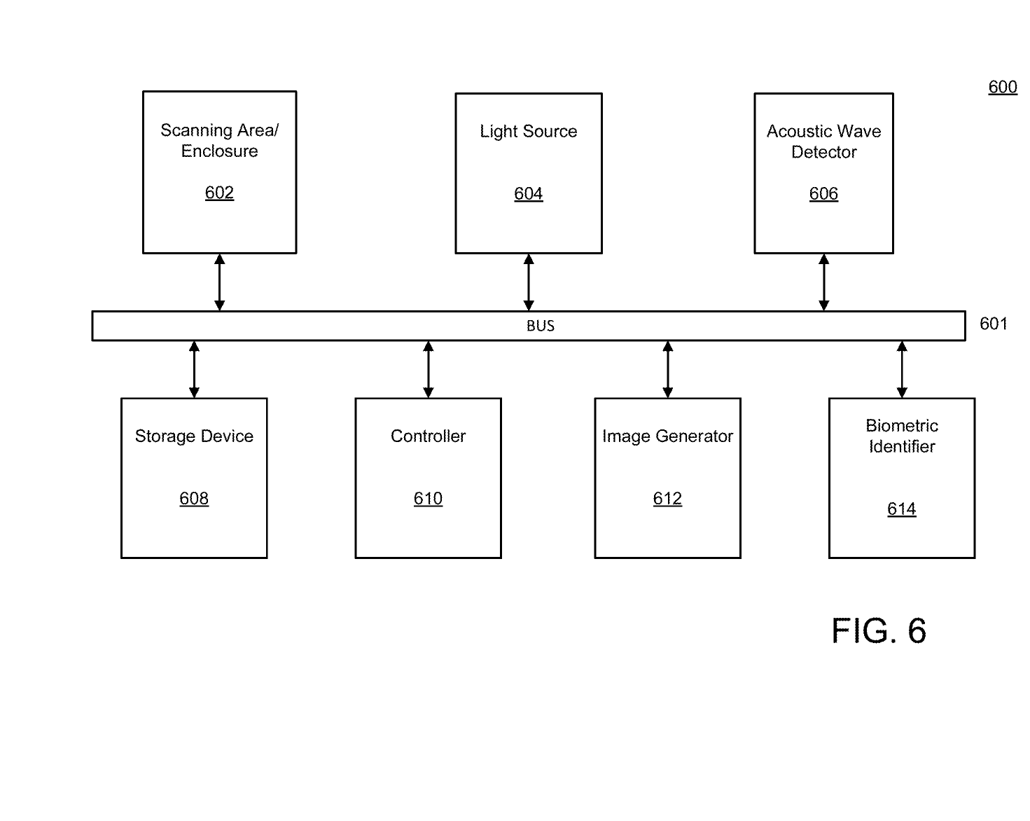

Referring now to FIG. 6, an exemplary system 700 for three-dimensional imaging of finger veins using photo-acoustic tomography for three-dimensional biometrics is illustratively depicted in accordance with the present principles.

While many aspects of system 600 are described in singular form for the sakes of illustration and clarity, the same can be applied to multiples ones of the items mentioned with respect to the description of system 600. For example, while a single light source 604 is described, more than one light source 604 can be used in accordance with the teachings of the present principles, while maintaining the spirit of the present principles. Moreover, it is appreciated that the light source 604 is but one aspect involved with system 600 than can be extended to plural form while maintaining the spirit of the present principles.

The system 600 can include a bus 601, a scanning area/enclosure 602 (e.g., for placement of biometric object to be imaged), a light source 604, and an acoustic wave detector 606. A storage device 608 (e.g., non-transitory computer readable medium) may be employed to store, for example, biometric data, acoustic waves, etc. A controller 610 (e.g., local or remote) may be employed to provide commands to the system 600, and an image generator 612 may output a three-dimensional image of a biometric object (e.g., finger veins) with no deleterious light scattering in accordance with the present principles. A biometric identification device 614 may be employed to verify an identity based on the three-dimensional image generated for use as a three-dimensional biometric in accordance with various embodiments of the present principles.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the principles of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A computer implemented method for imaging a three-dimensional (3D) biometric object, comprising:
receiving the 3D biometric object into a scanning area;
generating an image of the 3D biometric object using photo-acoustic tomography, the generating further comprising:
illuminating one or more veins in the 3D biometric object using a light source;
detecting acoustic waves resulting from absorption of light from the light source by the one or more veins; and
outputting a 3D vein image based on the acoustic waves detected.

2. The method of claim 1, wherein the 3D biometric object is at least one of a finger, palm, arm, leg, or eye.

3. The method of claim 1, wherein the light source and the acoustic wave detector are positioned on a same side of the 3D biometric object, on opposite sides of the 3D biometric object, at a right angle to each other, or at an arbitrary angle to each other.

4. The method of claim 1, wherein the light is a light pulse or continuous wave light comprising a wavelength of light absorbed by blood in the one or more veins.

5. The method of claim 1, wherein the light source is one of a pulsed laser, a temporally modulated continuous wave laser, or a temporally modulated non-laser.

6. The method of claim 1, wherein the light source has spatial resolution, has no spatial resolution, or is raster scanned.

7. The method of claim 2, wherein the acoustic wave detector is in contact with the 3D biometric object.

8. The method of claim 2, wherein the acoustic wave detector is in non-contact with the 3D biometric object.

9. The method of claim 1, wherein the acoustic wave detector has spatial resolution, has no spatial resolution, or is raster scanned.

10. A system for imaging a three-dimensional (3D) biometric object, comprising:
a scanning area for receiving the 3D biometric object for imaging; and
an image generator for generating an image of the 3D biometric object using photo-acoustic tomography, the image generator being further configured to:
illuminate one or more veins in the 3D biometric object using a light source;
detect acoustic waves resulting from absorption of light from the light source by the one or more veins using an acoustic wave detector; and
output a 3D vein image based on the acoustic waves detected.

11. The system of claim 10, wherein the 3D biometric object is at least one of a finger, palm, arm, leg, or eye.

12. The system of claim 10, wherein the light source and the acoustic wave detector are positioned on a same side of the 3D biometric object, on opposite sides of the 3D biometric object, at a right angle to each other, or at an arbitrary angle to each other.

13. The system of claim 10, wherein the light is a light pulse or continuous wave light comprising a wavelength of light absorbed by blood in the one or more veins.

14. The system of claim 10, wherein the light source is one of a pulsed laser, a temporally modulated continuous wave laser, or a temporally modulated non-laser.

15. The system of claim 10, wherein the light source has spatial resolution, has no spatial resolution, or is raster scanned.

16. The system of claim 11, wherein the acoustic wave detector is in contact with the 3D biometric object.

17. The system of claim 11, wherein the acoustic wave detector is in non-contact with the 3D biometric object.

18. The system of claim 10, wherein the acoustic wave detector has spatial resolution, has no spatial resolution, or is raster scanned.

19. A non-transitory computer-readable storage medium comprising a computer-readable program for imaging a three-dimensional (3D) biometric object, wherein the computer-readable program when executed on a computer causes the computer to perform the steps of:
receiving the 3D biometric object into a scanning area;
generating an image of the 3D biometric object using photo-acoustic tomography, the generating further comprising:
illuminating one or more veins in the 3D biometric object using a light source;
detecting acoustic waves resulting from absorption of light from the light source by the one or more veins; and
outputting a 3D vein image based on the acoustic waves detected.

20. The computer-readable storage medium of claim 19, wherein the light is a light pulse or continuous wave light comprising a wavelength of light absorbed by blood in the one or more veins.
